(19) United States
(12) Patent Application Publication
(10) Pub. No.: US 2018/0052142 A1
(43) Pub. Date: Feb. 22, 2018

(54) Method and Apparatus for Visually and Electrically Detecting Dissolved Hydrogen Gas in Liquids

(71) Applicants: William Hoagland, Boulder, CO (US); David K. Benson, Golden, CO (US); Rodney D. Smith, Golden, CO (US)

(72) Inventors: William Hoagland, Boulder, CO (US); David K. Benson, Golden, CO (US); Rodney D. Smith, Golden, CO (US)

(21) Appl. No.: 15/672,184

(22) Filed: Aug. 8, 2017

Related U.S. Application Data
(60) Provisional application No. 62/372,175, filed on Aug. 8, 2016.

(51) Int. Cl. G01N 33/00; G01N 21/78; G01N 27/04

(52) U.S. Cl. CPC G01N 33/005; G01N 2021/7796; G01N 27/04; G01N 21/783

(57) ABSTRACT

Element One has developed thin films have the ability to quickly, reliably and cost-effectively detect the presence of dissolved hydrogen in liquids through either a visible color change or a measurable resistance change. Thin film sensors are multi-layer thin film devices incorporating a substrate, an active transition metal oxide layer, a discontinuous catalyst layer, as necessary nn additional protective layer. This invention may be used for early detection of fault conditions in transformer oils allowing low cost tests that can effectively Such thin films can also detect the presence of dissolved hydrogen in aqueous solutions for certain therapeutic heath drinks and other applications.

### CROSS-REFERENCE TO RELATED APPLICATIONS

This application claims the benefit of provisional patent applications Ser. No. 62/372,175, filed 2016 Aug. 9 by the present inventors.

### FEDERALLY SPONSORED RESEARCH

Development work in support of this invention was provided by the U.S. Department of Energy through the National Renewable Energy Laboratory, Golden, Colo., subcontract LHL-6-62582-01.

The following is a tabulation of some prior art that presently appears to be relevant:

### RELEVANT PRIOR ART INCLUDES

### U.S. Patent

U.S. Pat. No. 6,895,805B2—HYDROGEN GAS INDICATOR SYSTEM—Hoagland, W., May 24, 2005

U.S. Pat. No. 8,636,883B2—MONITORABLE HYDROGEN SENSOR SYSTEM—Hoagland, W., Benson, D. K., Jan. 28, 2014

### REFERENCES CITED

U.S. Pat. No. 9,377,2016—SENSOR ASSEMBLY AND METHOD FOR SENSING STATUS CONDITION OF ELECTRICAL EQUIPMENT—Panella, et al, Jun. 28, 2016

### BACKGROUND

There are numerous industrial operations in which the presence or the concentration of dissolved hydrogen gas is important.

One example is where electrical insulation of coil windings in high-voltage electrical power transformers degrades over time allowing electrical discharges to occur within the housing. The housing is filled with organic or silicone polymer oil to dissipate the internal heat. The electrical discharges decompose some of the oil and produces dissolved hydrogen and other decomposition gases. Large power transformers are filled with oil that cools and insulates the transformer windings; this insulating liquid is in contact with the internal components, and gases formed by normal and abnormal events within the transformer are dissolved in the oil. These gases are produced by oxidation, vaporization, insulation decomposition, oil breakdown and electrolytic action.

The analysis of gases from petroleum products has been performed for decades using gas chromatography. However, this technique was not applied specifically to transformer mineral oil until the late 1960s/early 1970s and is now commonly called dissolved gas-in-oil analysis (DGA). Analyzing the volume, types, proportions, and rate of production of dissolved “fault gases,” can reveal the faults of a transformer. Off line dissolved gas analysis (DGA) techniques require oil samples to be taken for laboratory evaluation, while online transformer monitoring generally has been constrained to transmission-class components due to the requirement for sophisticated and costly analytical equipment.

Hydrogen gas is a key gas that is produced in all the three of most common transformer faults. Since the current methods of Dissolved Gas Analysis require taking a sample of the transformer oil and transporting it to a laboratory, this method is inconvenient and costly. As a result, this type of analysis is performed on only the larger power transformers on a regular basis, usually once per year. Furthermore, such transformers are installed in an electrical grid where there are many interlocks that can shut down a transformer when a problem is detected. However this shutdown does not always indicated a transformer fault but can also be cause by grid aberrations independent of a particular transformer. When a safety interlock shuts down a transformer, it usually requires a dissolved gas analysis before going back online. If there were a simple, low-cost, rapid test available to rule out serious faults, it could be possible to put the transformer back in service immediately with minimal shutdown time.

Currently, industry practice requires that the transformer oil be routinely sampled and tested by detailed laboratory chemical analyses. Element One's low cost test has proven an effective tool to screen for the presence of hydrogen and, in the absence of hydrogen, forgo the expensive laboratory testing. Element One has provided the first 5,000 test kits to NextEra, Inc. and their test results to date have proved that this is an effective alternative to the routine detailed laboratory testing or expensive online testing.

The concentration of the dissolved hydrogen gas is a useful indicator of the degree of degradation of the transformer coil insulation and may be used to indicate when the transformer is in need of repair or at risk of failure. Transformer failures cause costly power outages, fires and explosions. There is a well-known and urgent need to detect coil insulation degradation before such a catastrophic failure occurs. Monitoring the occurrence and/or the concentration of dissolved hydrogen gas in the transformer oil is a possible means to detect and monitor such insulation degradation

In previous research, Element One has developed several thin film indicators which change color and conductivity to indicate the presence of hydrogen gas for leak detection (U.S. Pat. No. 6,895,805). “HYDROGEN INDICATOR SYSTEM, May 4, 2005). Although it might be obvious to one skilled in the art, this application recognizes the ability to detect hydrogen in liquids such as oils and aqueous solutions.

In preliminary tests, Element One has found that this technology can also quickly, reliably and cost-effectively detect the presence of dissolved hydrogen in transformer oils to screen for the existence of fault conditions. Element One has been working with industry to develop and test a thin film dissolved hydrogen indicators for application to for safety inspection of high voltage power transformers. As transformers age, the insulation on the windings degrade and electrical arcing can occur, this arcing decomposes the transformer oil into several degradation products including hydrogen gas. By checking the transformer oil for dissolved hydrogen, the inspector can determine whether the transformer has degraded enough to require more extensive testing or removal from service.

Another example is to test for the presence of dissolved hydrogen the cooling water in a turbine electrical power generator. The rotating coils of the generator must be cooled. Pressurized hydrogen gas is commonly used as a heat transfer medium between the rotating coils and a water-cooled stationary jacket. If a leak occurs between the hydrogen blanket and the water-cooled jacket, hydrogen gas can dissolve in the cooling water. Cooling water leaking onto the rotating coils can cause an expensive and dangerous catastrophic failure of the generator. There is a well-known and urgent need to detect such early signs of leaks. Detecting the dissolved hydrogen in the cooling water may provide a convenient, early indication of the leak before it becomes a serious safety problem.

Another example is to test for dissolved hydrogen in therapeutic health drinks. Research has shown that consuming water with dissolved hydrogen can provide therapeutic benefit to patients with a wide range of ailments or medical conditions including Parkinson's disease and other neurological disorders associated with aging. Several methods have been studied, but the most practical way to do this is to ingest water containing between 5-15 parts per million dissolved hydrogen. Several companies have recently introduced such products to the market. However, the incorporation of dissolved hydrogen into consumer packaging is problematic in that molecular hydrogen gas dissolved in water is tasteless, invisible and odorless. In addition, the gas readily leaves the liquid when exposed to air and the containment of molecular hydrogen in cost effective consumer packaging is difficult; the gas tends to leak out of the container in a much shorter time that one sees with, for example, carbonated beverages. Therefore, there is a recognized need to be able to reassure the customer of the molecular hydrogen content at the point of purchase. This may be achieved by a visual hydrogen detector incorporated into the packaging showing that it retains its therapeutic value. Several configuration have been tested by Element One.

Although a patent search revealed significant prior art to measure gaseous hydrogen in a gaseous environment, as well as measuring dissolved hydrogen in oils, all prior art required more complicated processes such as first extracting the gas from the liquid, electronic sensing elements, optically coupled sensors, or chromatographic techniques. Our sensor is unique in that it will operate with no external power source making is suitable for integration with passive RFID technologies. For example, U.S. Pat. No. 9,377,451 “Sensor Assembly and method for sensing status condition of electrical equipment” Panella, et al. incorporates a hydrogen sensing element and an optical sensor with the related electronics. The subject invention is much simpler and easier to use at a fraction of the cost.

### SUMMARY OF THE INVENTION

The subject invention is a sensing element consisting of a multi-layer thin film coating containing a metal oxide chemotronic material (a material whose electronic properties change in the presence of a particular chemical) with a thin coating of a catalyst and a hydrogen permeable protective coating of polymer or ceramic that changes color in the presence of hydrogen gas. The metal oxide layer of said thin film also undergoes a large change in conductivity. These changes are chemically driven and do not require an external source of power and may or may not be connected to the electrical circuit of a wired or wireless measurement or communication device. The sensing element is immersed in the liquid. Dissolved hydrogen permeates through the protective, hydrogen-permeable coating and is dissociated into atomic hydrogen by the catalyst. The atomic hydrogen then reacts with the underlying layer of chemotronic metal oxide to chemically reduce it. This causes a visible color change in the metal oxide layer as well as a significant change in resistance. Whereas the fully oxidized chemotronic material is an electrical insulator, the partially reduced chemotronic material is a semiconductor with an electrical resistance that decreases as the metal oxide is chemically reduced. Whereas both the visual color and electrical resistance of the metal oxide layer changes, the reduction in the electrical resistance of the multi-layer thin film coating may be used to signal the presence of hydrogen gas in the liquid. The total extent of the chemical reduction of the chemotronic metal oxide and/or the rate of the chemical reduction may be used to indicate the concentration of the dissolved hydrogen as well as its presence.

### ADVANTAGES

Accordingly several advantages of one or more aspects are as follows: to provide a low-cost reliable indication of the presence of dissolved hydrogen in liquids that does not require external power, is convenient to use, and may be used in the area of use by a plant operator or consumer. The design of the subject invention permits the production of a very low cost device that may be economically used pervasively at sites where knowledge of the presence of, or concentration of, dissolved hydrogen gas is important. The design of the subject invention permits its use in visual indicators, wired or wireless sensor networks without the requirement of electrical heating and, consequently, without the need for much continuous electrical power consumption.

The design of the subject invention permits the production of a very low cost device that may be economically used pervasively at sites where knowledge of the presence of, or concentration of, dissolved hydrogen gas is important. The design of the subject invention permits its use in visual indicators, wired or wireless sensor networks without the requirement of electrical heating and, consequently, without the need for much continuous electrical power consumption.

### BRIEF DESCRIPTION OF SEVERAL VIEWS OF THE DRAWING

FIG. 1 shows the cross section of a sensor for detecting dissolved hydrogen gas in liquids.

FIG. 2 shows a typical wireless circuit schematic that could be used in conjunction with the hydrogen sensor. There are many possible circuit designs possible for this application

FIG. 3 shows an example of a single use visual sensor where the protective system is a glass vial. Inside the vial, a clear thin film tab can be clearly seen to darken in the presence of dissolved hydrogen

### DETAILED DESCRIPTION OF THE INVENTION

The sensor consists of an electrically insulating substrate on which a multi-layer thin film stack has been deposited. The thin film stack includes a layer of a metal oxide chemotronic material such as tungsten oxide, molybdenum oxide, vanadium oxide, palladium oxide or the like with a discontinuous coating of a catalytic material such as platinum, palladium, rhodium, cerium oxide, or the like and a hydrogen permeable protective polymer coating (See FIG. 1). When dissolved hydrogen gas permeates the protective hydrogen-permeable coating, it is dissociated by the catalyst into atomic hydrogen. The atomic hydrogen will partially reduce the chemotronic metal oxide. Whereas the fully oxidized chemotronic material is an electrical insulator, the partially reduced chemotronic material is a semiconductor with an electrical resistance that depends upon the degree of its chemical reduction.

A typical embodiment of the subject dissolved hydrogen sensor includes a film of nano-porous tungsten oxide of thickness 350 nanometers deposited upon an insulating substrate. A 3 nanometer thick layer of metallic palladium is applied on top of the tungsten oxide. A 100 nanometers coating of nano-porous polytetrafluoroethylene (PTFE) is applied as a hydrogen-permeable protective layer over the palladium. An additional hydrogen-permeable polymer such as silicone or the like may be applied as a further protective covering over the sensor. Other metals, metal oxides and permeable protective coatings may be used as would be obvious to anyone skilled in the art.

Metallic electrical connections to the thin film sensor allow a low voltage to be applied to it. These electrical connections may take the form of metal foils or evaporated metal strips or other such means applied to either end of a length of the multi-layer thin film device or they may take the form of an array of interdigitated contacts applied to one of the surfaces of the thin chemotronic film.

### Operation

The subject dissolved hydrogen gas sensor may be immersed into the oil of a high-voltage power transformer and either visually inspected or electrically connected to a wired or wireless communication network to communicate the occurrence and/or the concentration of dissolved hydrogen in the transformer oil as a sign of degradation of the transformer coil insulation.

The dissolved hydrogen gas sensor element is a hydrogen sensitive resistor whose resistance decreases in the presence of hydrogen gas. The electrical current that flows through the chemotronic film is inversely proportional to the film resistance. As hydrogen is dissociated into atomic hydrogen by the catalyst, it then reacts with the chemotronic film to decrease its resistance. The electrical current in the sensor increases. It is this increase in current that indicates the decrease in film resistance and the corresponding presence of dissolved hydrogen gas. Typically, the sensor would be connected to a wireless or wired measurement or communication device and the sensor status would be communicated to a monitoring location.

It is well known that the microprocessor of a communications node designed for use with sensors has a circuit which can be programmed to measure the characteristics of an attached sensor device. In the case of the hydrogen sensor, the microprocessor is programmed to measure the resistance of the attached hydrogen sensitive resistor.

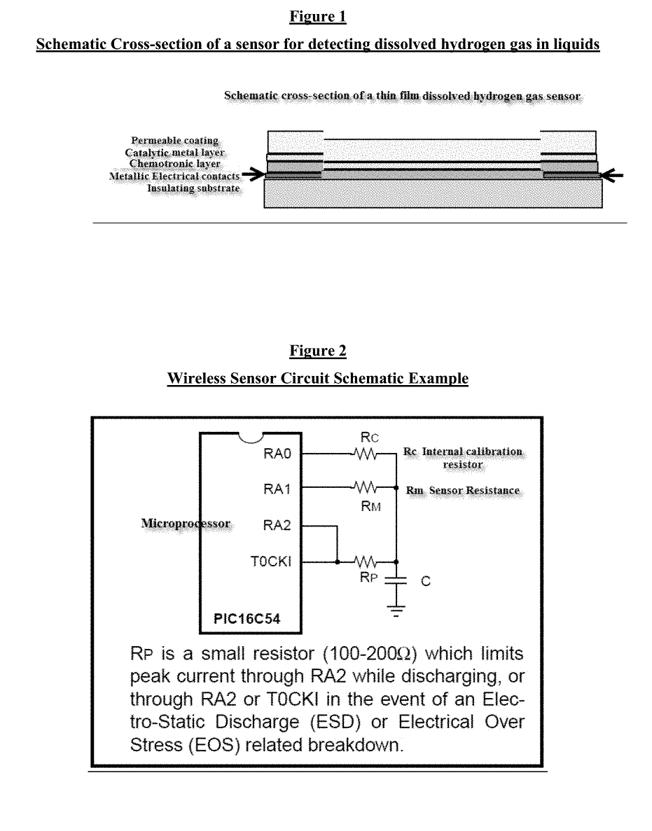

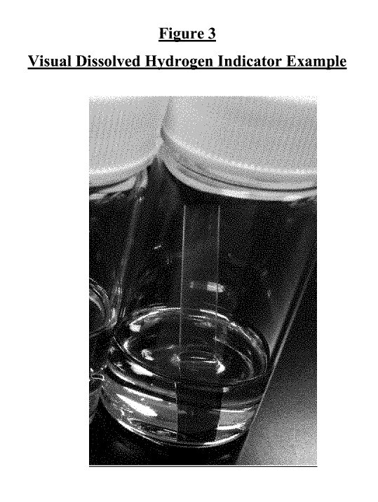

One of such typical circuits for measuring the resistance of an external device has been described by D. Cox in an application note, “Implementing Ohmmeter/Temperature Sensor” published by Microchip Technology Inc. and available at their interne web site (See FIG. 2): http://www.microchip.com/stellent/idcplg?IdcService=SS_GET_PAGE&nodeId=1824&appnote=en010996

The hydrogen sensitive resistor may be incorporated as a component in an attached resistor-capacitor (RC) circuit. In this particular design of a wireless sensor, the sensor derives its operational electrical power from an interrogation radio frequency broadcast. The interrogation radio frequency signal is received by the wireless sensor circuit and rectified to produce a voltage, a portion of which is applied to the RC circuit. After the voltage is momentarily applied to the capacitor of the RC circuit the charge on the capacitor leaks off through the hydrogen sensitive resistor, Rm. The time required for the voltage on the capacitor to drop from the initial value to a lower reference value is inversely proportional to the resistance in the RC circuit. The microprocessor is programmed to count the number of internal clock cycles that elapse between the time the voltage is initially applied and the time the voltage reaches a reference value. It then triggers a radio frequency emitter circuit and transmits the sensor's identifying code and the count of elapsed clock cycles. If the count is significantly lower than normal, the signal indicates that this particular sensor has detected hydrogen gas. The greater the decrease in count number, the stronger is the indication. If the responses are collected over a period of time, the rate of decrease in count number could be used to indicate the concentration of hydrogen gas at the sensor.

### Alternative Embodiments

The subject invention may be constructed with any of the listed chemochromic metal oxides and catalytic materials in a wide range of film thicknesses. Other metal oxides and catalytic materials in addition to those listed may also be used as would be obvious to anyone skilled in the art.

Many protective polymer or porous ceramic coatings may be used as an alternative to the PTFE as would be obvious to anyone skilled in the art.

A wide range of electronic measurement and communication circuits, both wired and wireless, with connections for monitoring the resistance of an internal or external sensor device are well known and may be used with the subject invention.

The subject invention may be constructed on a solid substrate material and with no protective coating over the hydrogen sensitive material and enclosed in a suitable sealable protective container to which a liquid sample can be added (See FIG. 3). In this embodiment, with no protective coating, the hydrogen sensitive chemochromic material will have increased sensitivity to dissolved molecular hydrogen and will visually change color in the presence of dissolved molecular hydrogen at concentrations of interest.

The subject invention may also be inserted into a ˜15 ml glass vial and sealed for protection prior to use. A sample of the hydrogen containing liquid, e.g., transformer oil could be inserted into vial. The vial is recapped and shaken gently. After 3-10 minutes the color change of the sensor will be observed if dissolved hydrogen is present. The darker the color change, the more dissolved hydrogen is present. This test can be referenced against known samples with regard to the amount and speed of the color change to determine of the amount of hydrogen is at an actionable level. Only those samples which show actionable levels will need to be sent for further testing such as DGA.

The embodiments of the invention in which an exclusive property or privilege is claimed are identified as follows:

1. A hydrogen detector apparatus comprising: a strip of material including an electrically insulating substrate; a set of at least two electrically conductive contacts; a thin film of hydrogen sensitive material whose electrical resistance changes in the presence of hydrogen; a discontinuous thin film of a catalyst in contact with the hydrogen sensitive material; and a protective coating over the hydrogen sensitive material and catalyst.

2. A hydrogen detector apparatus of claim 1, wherein the electrically insulating substrate is a ceramic or glass sheet.

3. A hydrogen detector apparatus of claim 1, wherein the electrically insulating substrate is a plastic sheet.

4. A hydrogen detector apparatus of claim 1, wherein the electrically conductive contacts are arranged in an interdigitated array with the hydrogen sensitive material bridging over the array so as to complete an electrical path between the first electrical contact and the second electrical contact.

5. A hydrogen detector apparatus of claim 1, wherein the electrically conductive contacts are made from thin films of gold, silver, copper or other highly conductive material.

6. A hydrogen detector apparatus of claim 1, wherein the electrically conductive contacts are made of indium-tin oxide, arsenic-doped tin oxide, silicon, or a similarly conductive semiconductor material.

7. A hydrogen detector apparatus of claim 1, wherein the hydrogen sensitive material is a thin film of a transition metal oxide such as molybdenum trioxide, tungsten trioxide, vanadium pentoxide, niobium trioxide, palladium oxide or chromium trioxide.

8. A hydrogen detector apparatus of claim 1, wherein the hydrogen sensitive material is a thin film of thickness between 100 and 1000 nanometers thick.

9. A hydrogen detector apparatus of claim 1, wherein the catalyst thin film is made from a precious metal or combination of precious metals such as palladium, platinum, rhodium, or a mixed catalyst such as platinum-ruthenium (50%-50%) mixture.

10. A hydrogen detector apparatus of claim 1, wherein the catalyst film is discontinuous and has a thickness between 1 and 100 nanometers.

11. A hydrogen detector apparatus of claim 1, wherein the protective coating is hydrogen permeable; but resistant to water and other liquids.

12. A hydrogen detector apparatus of claim 1, wherein the protective coating is comprised of a polymer or polymer mixture such as silicone, polyethylene, polypropylene, nylon, poly acetate or other electrically insulating polymer.

13. A hydrogen detector apparatus of claim 1, wherein the protective coating is comprised of a hydrogen permeable ceramic or glass.

14. A hydrogen detector apparatus of claim 1, wherein the protective coating is between 10 and 500 microns thick.

15. A hydrogen detector apparatus of claim 1 incorporated into an electrical circuit which produces a visual readout or radio-frequency transmission of the indicator's electrical resistance.

16. A single-use visual hydrogen gas indicator that will change color in the presence of dissolved molecular hydrogen, comprising:
a. a substrate material;
b. an atomic hydrogen gas sensor supported by said substrate material wherein said atomic hydrogen gas sensor is selected from the group consisting of vanadium oxide, tungsten oxide, molybdenum oxide, yttrium oxide, palladium oxide and combinations thereof;
c. a catalyst material coupled to said atomic hydrogen gas sensor, wherein said catalyst material converts molecular hydrogen gas to atomic hydrogen gas sensed by said atomic hydrogen gas sensor; and
d. a sealable, protective system to prevent contamination of the active sensor materials prior to use that is suitable for depositing liquids of interest (e.g. transformer oil) to come in contact with the active sensor materials.

17. A hydrogen gas indicator as described in claim 16, wherein said catalyst material is selected from the group consisting of platinum, palladium, ruthenium, rhodium, nickel, and alloys of these materials with other metals.

18. A hydrogen gas indicator as described in claim 16, wherein said substrate material comprises a material that may include, but not be limited to PET.

19. A hydrogen gas indicator as described in claim 16, wherein said discrete indicia operably responsive to said atomic hydrogen gas sensor comprises symbols or characters applied to a surface which becomes visible in the presence of dissolved molecular hydrogen.

20. A hydrogen gas indicator as described in claim 16 where said protective system is comprised of a clear container such as a glass or plastic vial or eye dropper that can be filled with a liquid to be tested and observed for a color change.
